(19) United States
(12) Patent Application Publication
(10) Pub. No.: US 2024/0299349 A1
(43) Pub. Date: Sep. 12, 2024

(54) TREATMENT OR PREVENTION OF LEUKAEMIA

(71) Applicant: VESTLANDETS INNOVASJONSSELSKAP AS, Bergen (NO)

(72) Inventors: Hans-René BJØRSVIK, Bergen (NO); Bjørn Tore GJERTSEN, Morvik (NO)

(21) Appl. No.: 17/768,631

(22) PCT Filed: Oct. 14, 2020
(86) PCT No.: PCT/GB2020/052572

§ 371 Date: Apr. 13, 2022

(30) Foreign Application Priority Data
Oct. 14, 2019 (GB) 1914848.5

(51) Int. Cl. A61K 31/403; A61K 31/09; A61K 31/136

(52) U.S. Cl. CPC A61K 31/403; A61K 31/09; A61K 31/136

(57) ABSTRACT

The present invention provides compounds for use in the treatment or prevention of leukaemia which are based on a 2-amino-[1,1′]-biphenyl or corresponding carbazole scaffold, in particular, it provides the following compounds of formula (I), their stereoisomers, and their pharmaceutically acceptable salts for use in such treatment: (I) (I) wherein: Y is selected from C=0 and —CR⁹ (in which R⁹ is H, —OH or —O-alkyl, (e.g., —O—C₁₋₆ alkyl)); Z is selected from C=0 and —CR¹⁰ (in which R¹⁰ is H, —OH, —O-alkyl, (e.g., —O—C₁₋₄ alkyl) or —O—C(O)R^{A} wherein R^{A} is H or alkyl (e.g. C₁₋₄ alkyl)); R¹ is —NO₂ or —NR¹¹R¹² (wherein R¹¹ and R¹² are both H, or R¹¹ is H and R¹² is a group of the formula —C(O)R^{A} in which R^{A} is H or alkyl (e.g. C₁₋₆ alkyl)); each of R², R⁴, and R⁶ to R⁸ are independently selected from H, alkyl (e.g. C₁₋₆ alkyl), —O-alkyl (e.g. —O—C₁₋₆ alkyl), and halogen; R³ and R⁵ are independently selected from H, —O-alkyl (e.g. —O—C₁₋₆ alkyl), and halogen; or R¹ and R⁸ together form a group NR¹³ (in which R¹³ is H, alkyl (e.g. C₁₋₆ alkyl), or a group of the formula —C(O)R^{A} in which R^{A} is H or alkyl (e.g. G₁₋₆ alkyl)); and represents an optional bond between two adjacent carbon atoms in the ring; with the proviso that when Y and Z are both C═O, and R¹ and R⁸ together form a group NR¹³, at least one of R² and R³ is other than H or —CH₃. Such compounds find particular use in the treatment or prevention of chronic myeloid leukaemia (GML), acute myeloid leukaemia (AML), acute lymphocytic leukaemia (ALL) or t-ALL (T-cell acute lymphoblastic leukaemia).

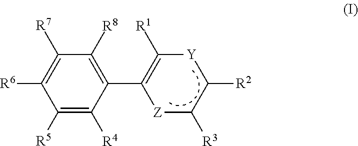

### FIELD OF THE INVENTION

The present invention relates to the treatment of leukaemia. In particular, it relates to the use of compounds based on a 2-amino-[1,1′]-biphenyl or corresponding carbazole scaffold, and derivatives thereof, in such treatment.

### BACKGROUND TO THE INVENTION

Leukaemia is a group of blood cancers that usually begin in the bone marrow and which result in high numbers of abnormal blood cells. There are four main types of leukaemia: acute lymphoblastic leukaemia (ALL), acute myeloid leukaemia (AML), chronic lymphocytic leukaemia (CLL) and chronic myeloid leukaemia (CML), as well as a number of less common types. Current treatment of leukaemia may involve a combination of chemotherapy, radiation therapy, targeted therapy and bone marrow transplant. In 2015, leukaemia was present in 2.3 million people and caused over 350,000 deaths. It is the most common type of cancer in children, with most of the leukaemia cases in children being of the ALL type. In adults, AML and CLL are the most common forms. There is a continuing need for other therapies for the treatment of leukaemia, in particular AML.

A variety of carbazole alkaloids, known as carbazomycin A-H (Scheme 1), have been discovered, isolated, and their structure elucidated. These have a range of biological activity including antibacterial, antifungal and anti-cancer properties (see, for example, Sakano et al., J. Antibiot. 1980, 33, 683-689; Kaneda et al., J. Antibiot. 1988, 41, 602-608; and Nishiyama et al., Eur. J. Med. Chem. 2016, 121, 561-577).

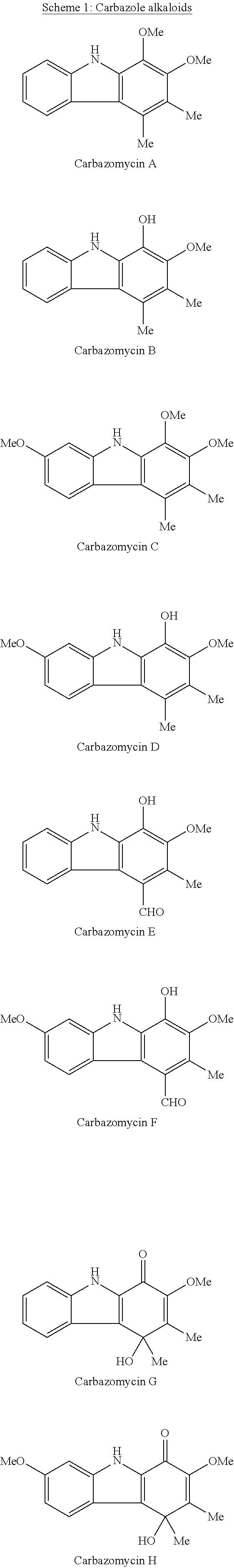

A series of carbazole alkaloids have also been isolated and investigated for their cytotoxic properties, including testing in the HL60 cell line which is used as a model for the exploration of new anti-leukaemic compounds (see Itoigawa et al., J. Nat. Prod. 2000, 63(7), 893-897; Ito et al., Phytomedicine 2006, 13, 359-365; and Ito et al., J. Nat. Med. 2012 66 357-361). Amongst the compounds tested are Murrayafoline A and Murrayaquinone A:

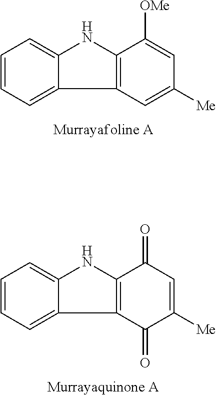 [SMILES: COc1cc(C)cc2c1[nH]c1ccccc12 ; CC1=CC(=O)c2[nH]c3ccccc3c2C1=O]

The carbazomycins (A-H) possess a peculiar substitution pattern in which one of the two aromatic rings that constitutes an integral part of the carbazole scaffold is unsubstituted or mono-substituted, while the other aromatic ring is substituted in all possible positions. The molecular structure of the carbazomycins, their range of biological activity and their peculiar substituted carbazole scaffold has made these an attractive molecular motif for the synthetic chemist and has resulted in the development of a number of total syntheses. One such strategy developed by the inventors is based on two distinct Pd-catalysed reactions. A Suzuki cross-coupling is performed to obtain a 2-nitro-1,1′-biphenyl intermediate that is reduced and protected. This is then subjected to a concomitant intramolecular C—H activation followed by a C—N bond formation that produces a congested carbazole intermediate. This 12-step total synthesis to the carbazole alkaloid carbazomycin G is described in Eur. J. Org. Chem., 2018, 1984-1992.

The inventors have now found that the intermediate 2-amino-[1,1′]-biphenyl and carbazole compounds produced in this 12-step total synthesis have cytotoxic effects. When investigated using in vitro tests involving the human cell lines HL60 and MOLM-13, which are models for acute myeloid leukaemia (AML), these have been found to be cytotoxic and, in some cases, have an IC₅₀ value of less than 10⁻⁴ M. This finding is surprising since the final natural product, carbazomycin G, was found not to exhibit cytotoxicity when tested in the same two cell lines.

The inventors thus now propose that these intermediate compounds produced in the synthesis of carbazomycin G can be used in the treatment and/or prevention of leukaemias, in particular AML.

### SUMMARY OF THE INVENTION

In one aspect, the invention relates to a compound of formula (I), a stereoisomer, or a pharmaceutically acceptable salt thereof, for use in the treatment or prevention of leukaemia:

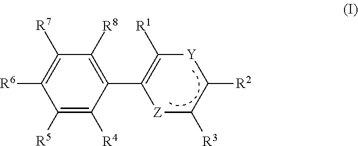

wherein:
Y is selected from C═O and —CR⁹ (in which R⁹ is H, —OH or —O-alkyl (e.g. —O—C₁₋₆ alkyl));
Z is selected from C═O and —CR¹⁰ (in which R¹⁰ is H, —OH, —O-alkyl (e.g. —O—C₁₋₆ alkyl) or —O—C(O)R^{A} wherein R^{A} is H or alkyl (e.g. C₁₋₆ alkyl));
R¹ is —NO₂ or —NR¹¹R¹² (wherein R¹¹ and R¹² are both H, or R¹¹ is H and R¹² is a group of the formula —C(O)R^{A} in which R^{A} is H or alkyl (e.g. C₁₋₆ alkyl));
each of R², R⁴, and R⁶ to R⁸ are independently selected from H, alkyl (e.g. C₁₋₆ alkyl), —O-alkyl (e.g. —O—C₁₋₈ alkyl), and halogen;
R³ and R⁵ are independently selected from H, —O-alkyl (e.g. —O—C₁₋₆ alkyl), and halogen;
or R¹ and R⁸ together form a group NR¹³ (in which R¹³ is H, alkyl (e.g. C₁₋₆ alkyl), or a group of the formula —C(O)R^{A} in which R^{A} is H or alkyl (e.g. C₁₋₆ alkyl)); and
[symbol] represents an optional bond between two adjacent carbon atoms in the ring;
with the proviso that when Y and Z are both C═O, and R¹ and R⁸ together form a group NR¹³, at least one of R² and R³ is other than H or —CH₃.

In a further aspect, the invention relates to a compound of formula (I), a stereoisomer, or a pharmaceutically acceptable salt thereof, for use in therapy or for use as a medicament.

In another aspect, the invention relates to a pharmaceutical composition comprising a compound of formula (I), a stereoisomer, or a pharmaceutically acceptable salt thereof, together with one or more pharmaceutically acceptable carriers, excipients or diluents.

In a further aspect the invention relates to the use of a compound of formula (I), a stereoisomer, or a pharmaceutically acceptable salt thereof, in the manufacture of a medicament for use in the treatment or prevention of leukaemia.

In another aspect, the invention relates to a method of treatment or prevention leukaemia, said method comprising the step of administering to a subject in need thereof (e.g. a human patient) a pharmaceutically effective amount of a compound of formula (I), a stereoisomer, or a pharmaceutically acceptable salt thereof.

### DETAILED DESCRIPTION OF THE INVENTION

### Definitions

As used herein, the term “alkyl” refers to a saturated hydrocarbon group and is intended to cover both straight-chained and branched alkyl groups. Examples of such groups include methyl, ethyl, n-propyl, iso-propyl, n-butyl, tert-butyl, sec-butyl, n-pentyl, iso-pentyl, neo-pentyl, n-hexyl, 2-methylbutyl, 2-methylpentyl, 2-ethylbutyl, 3-methylpentyl, and 4-methylpentyl. An alkyl group preferably contains from 1-6 carbon atoms, more preferably 1-4 carbon atoms, e.g. 1-3 carbon atoms. Unless otherwise specified, any alkyl group may be substituted in one or more positions with a suitable substituent. Where more than one substituent group is present, these may be the same or different. Suitable substituents include hydroxy, —O—C₁₋₃ alkyl, and halogen atoms.

The term “halogen” or “halogen atom” as used herein refers to —F, —Cl, —Br or —I.

The compounds herein described may contain one or more stereocenters and may therefore exist in different stereoisomeric forms. The term “stereoisomer” refers to compounds which have identical chemical constitution but which differ in respect of the spatial arrangement of the atoms or groups. Examples of stereoisomers are enantiomers and diastereomers. The term “enantiomers” refers to two stereoisomers of a compound which are non-superimposable mirror images of one another. The term “diastereoisomers” refers to stereoisomers with two or more stereocenters which are not mirror images of one another.

The invention is considered to extend to diastereomers and enantiomers, as well as racemic mixtures and enantioenriched mixtures in which the ratio of enantiomers is other than 1:1.

The compounds herein described may be resolved into their enantiomers and/or diastereomers. For example, where these contain only one chiral center, these may be provided in the form of a racemate or racemic mixture (a 50:50 mixture of enantiomers) or may be provided as pure enantiomers, i.e. in the R- or S-form. Any of the compounds which occur as racemates may be separated into their enantiomers by methods known in the art, such as column separation on chiral phases or by recrystallization from an optically active solvent. Those compounds with at least two asymmetric carbon atoms may be resolved into their diastereomers on the basis of their physical-chemical differences using methods known per se, e.g. by chromatography and/or fractional crystallization, and where these compounds are obtained in racemic form, they may subsequently be resolved into their enantiomers.

The term “pharmaceutically acceptable salt” as used herein refers to any pharmaceutically acceptable organic or inorganic salt of any of the compounds herein described. A pharmaceutically acceptable salt may include one or more additional molecules such as counter-ions. The counter-ions may be any organic or inorganic group which stabilises the charge on the parent compound. If the compound is a base, a suitable pharmaceutically acceptable salt may be prepared by reaction of the free base with an organic or inorganic acid. If the compound is an acid, a suitable pharmaceutically acceptable salt may be prepared by reaction of the free acid with an organic or inorganic base. Non-limiting examples of suitable salts are described herein.

The term “pharmaceutically acceptable” means that the compound or composition is chemically and/or toxicologically compatible with other components of the formulation or with the patient to be treated.

By “a pharmaceutical composition” is meant a composition in any form suitable to be used for a medical purpose.

As used herein, “treatment” includes any therapeutic application that can benefit a human or non-human animal (e.g. a non-human mammal). Both human and veterinary treatments are within the scope of the present invention, although primarily the invention is aimed at the treatment of humans. Treatment is intended to refer to the reduction, alleviation or elimination, preferably to normal levels, of one or more of the symptoms of the condition which is being treated relative to the symptoms prior to treatment. Where not explicitly stated, treatment encompasses prevention. As used herein, “prevention” refers to absolute prevention, i.e. maintenance of normal levels with reference to the extent or appearance of a particular symptom of the condition, or reduction or alleviation of the extent or timing (e.g. delaying) of the onset of that symptom.

As used herein, a “pharmaceutically effective amount” relates to an amount that will lead to the desired pharmacological and/or therapeutic effect, i.e. an amount of the agent which is effective to achieve its intended purpose. While individual subject (e.g. patient) needs may vary, determination of optimal ranges for effective amounts of the active agent(s) herein described is within the capability of one skilled in the art. Generally, the dosage regimen for treating a disease, condition or disorder with any of the compounds described herein may be selected by those skilled in the art in accordance with a variety of factors including the nature of the condition and its severity.

The term “subject” refers to any individual who is the target of the administration or treatment. The subject may be, for example, a mammal. Thus the subject may be a human or non-human animal. The term “patient” refers to a subject under the treatment of a clinician. Preferably, the subject will be a human.

In one aspect, the invention provides a compound of formula (I), a stereoisomer, or a pharmaceutically acceptable salt thereof, for use in the treatment or prevention of leukaemia:

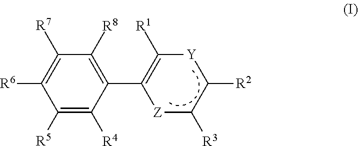

wherein:
Y is selected from C═O and —CR⁹ (in which R⁹ is H, —OH or —O-alkyl (e.g. —O—C₁₋₆ alkyl));
Z is selected from C═O and —CR¹⁰ (in which R¹⁰ is H, —OH, —O-alkyl (e.g. —O—C₁₋₆ alkyl) or —O—C(O)R^{A} wherein R^{A} is H or alkyl (e.g. C₁₋₆ alkyl));
R¹ is —NO₂ or —NR¹¹R¹² (wherein R¹¹ and R¹² are both H, or R¹¹ is H and R¹² is a group of the formula —C(O)R^{A} in which R^{A} is H or alkyl (e.g. C₁₋₆ alkyl));
each of R², R⁴, and R⁶ to R⁸ are independently selected from H, alkyl (e.g. C₁₋₆ alkyl), —O-alkyl (e.g. —O—C₁₋₆ alkyl), and halogen;
R³ and R⁵ are independently selected from H, —O-alkyl (e.g. —O—C₁₋₆ alkyl), and halogen;
or R¹ and R⁸ together form a group NR¹³ (in which R¹³ is H, alkyl (e.g. C₁₋₆ alkyl), or a group of the formula —C(O)R^{A} in which R^{A} is H or alkyl (e.g. C₁₋₆ alkyl)); and [symbol]
   represents an optional bond between two adjacent carbon atoms in the ring; with the proviso that when Y and Z are both C═O, and R¹ and R⁸ together form a group NR¹³, at least one of R² and R³ is other than H or —CH₃.

In one embodiment of formula (I), Y is —CR⁹ where R⁹ is as herein defined. In certain embodiments, R⁹ is —O-alkyl, preferably —O—C₁₋₆ alkyl, more preferably —O—C₁₋₃ alkyl, e.g. methyl.

In one embodiment of formula (I), Z is —CR¹⁰ where R¹⁰ is as herein defined. In certain embodiments, R¹⁰ is —OH or —O—C(O)R^{A} in which R^{A} is H or alkyl (e.g. C₁₋₆ alkyl), preferably C₁₋₃alkyl, e.g. methyl. In a preferred embodiment, Z is —C—OH.

In certain embodiments, the compounds for use in the invention are based on a biphenyl scaffold or corresponding carbazole. Such compounds are those of formula (I) in which Y is a group —CR⁹ and Z is a group —CR¹⁰. Thus, in one aspect, the compounds for use in the invention are those of formula (Ia), their stereoisomers, and pharmaceutically acceptable salts thereof:

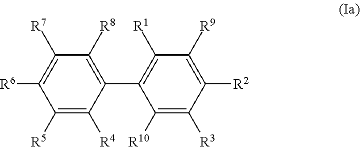

wherein R¹ to R¹⁰ are as herein defined.

In one embodiment of formula (Ia), R⁹ is —O-alkyl, preferably —O—C₁₋₆ alkyl, more preferably —O—C₁₋₃ alkyl, e.g. —O—CH₃.

In one embodiment of formula (Ia), R¹⁰ is selected from —OH and —O—C(O)R^{A} in which R^{A} is H or alkyl, preferably C₁₋₆ alkyl, more preferably C₁₋₃ alkyl, e.g. methyl. Preferably, R¹⁰ is OH.

In certain embodiments, one of the rings in the compounds for use in the invention is a quinone. Such compounds are those of formula (I) in which both Y and Z are C═O. Thus, in one aspect, the compounds for use in the invention are those of formula (Ib), their stereoisomers, and pharmaceutically acceptable salts thereof:

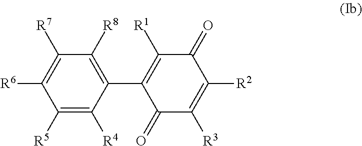

wherein R¹ to R⁸ are as herein defined, subject to the proviso that when R¹ and R⁸ together form a group NR¹³, at least one of R² and R³ is other than H or —CH₃.

In certain embodiments, the compounds for use in the invention are those of formula (I), (Ia) or (Ib) in which R¹ is —NR¹¹R¹² wherein R¹¹ and R¹² are both H, or R¹¹ is H and R¹² is a group of the formula —C(O)R^{A} (wherein R^{A} is H or alkyl, preferably C₁₋₆ alkyl, more preferably C₁₋₃ alkyl, e.g. methyl). In one embodiment, R¹ is —NH₂. Such compounds include those of formula (Ia′):

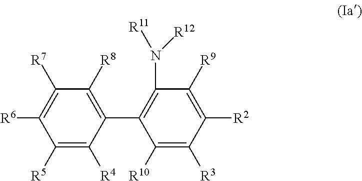

wherein each of R² to R¹² are as herein defined.

In certain embodiments of any of the compounds herein described, R⁸ is H.

In other embodiments, the compounds for use in the invention are those in which R¹ and R⁸ together form a group NR¹³ (in which R¹³ is as herein defined, preferably wherein R¹³ is H, or a group of the formula —C(O)R^{A} in which R^{A} is H or alkyl, preferably C₁₋₆ alkyl, more preferably C₁₋₃ alkyl, e.g. methyl). Such compounds include those of formula (Ia″):

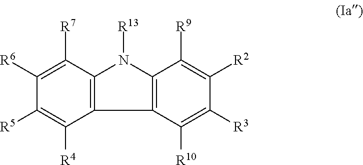

wherein each of R² to R⁷, R⁹, R¹⁰ and R¹³ are as herein defined.

In certain embodiments of any of the compounds herein described, R¹³ is H. In other embodiments, R¹³ is a group of the formula —C(O)R^{A} in which R^{A} is H or alkyl, preferably C₁₋₆ alkyl, more preferably C₁₋₃ alkyl, e.g. methyl.

In certain embodiments of any of the compounds herein described, R² is alkyl, preferably C₁₋₈ alkyl, more preferably C₁₋₃ alkyl, e.g. methyl.

In certain embodiments of any of the compounds herein described, R³ is —O—C₁₋₆ alkyl, preferably —O—C₁₋₆ alkyl, more preferably —O—C₁₋₃ alkyl, e.g. —OCH₃.

In certain embodiments of any of the compounds herein described, R⁴, R⁶ and R⁷ are each H.

In certain embodiments of any of the compounds herein described, R⁵ is H or —O-alkyl, preferably —O—C₁₋₈ alkyl, more preferably —O—C₁₋₃ alkyl, e.g. —O—CH₃.

Examples of compounds for use in accordance with the invention include, but are not limited to, the following and their pharmaceutically acceptable salts:

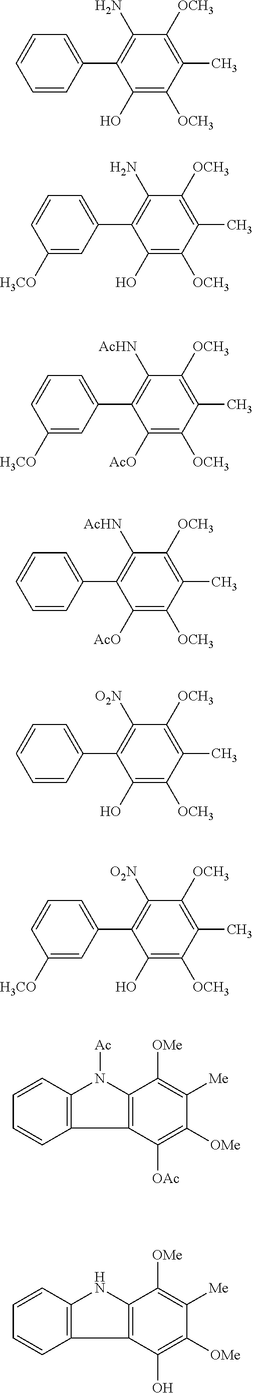

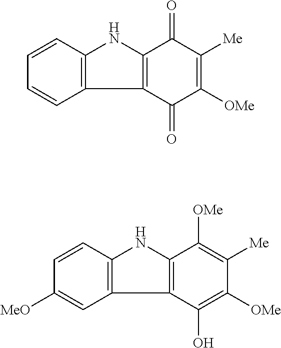 [SMILES: COC1=C(C)C(=O)c2[nH]c3ccccc3c2C1=O ; COc1ccc2[nH]c3c(OC)c(C)c(OC)c(O)c3c2c1]

The compounds for use in the invention are either known in the art, or can be prepared by methods known to those skilled in the art using readily available starting materials. Any of the compounds which are not known in the art may be prepared from readily available starting materials using known synthetic methods such as those described in known textbooks, for example, in Advanced Organic Chemistry (March, Wiley Interscience, 5^{th} Ed. 2001) or Advanced Organic Chemistry (Carey and Sundberg, KA/PP, 4^{th} Ed. 2001). Any compounds which are not known in the art form a further aspect of the invention.

The following scheme illustrates a general method for preparing the compounds herein described. Further details relating to this method are described in Elumalai et al., Eur. J. Org. Chem. 2018, 1984-1992, the entire content of which is incorporated herein by reference. The compounds used as starting materials are either known from the literature or may be commercially available. As will be understood, other synthetic routes may be used to prepare the compounds using different starting materials, different reagents and/or different reaction conditions.

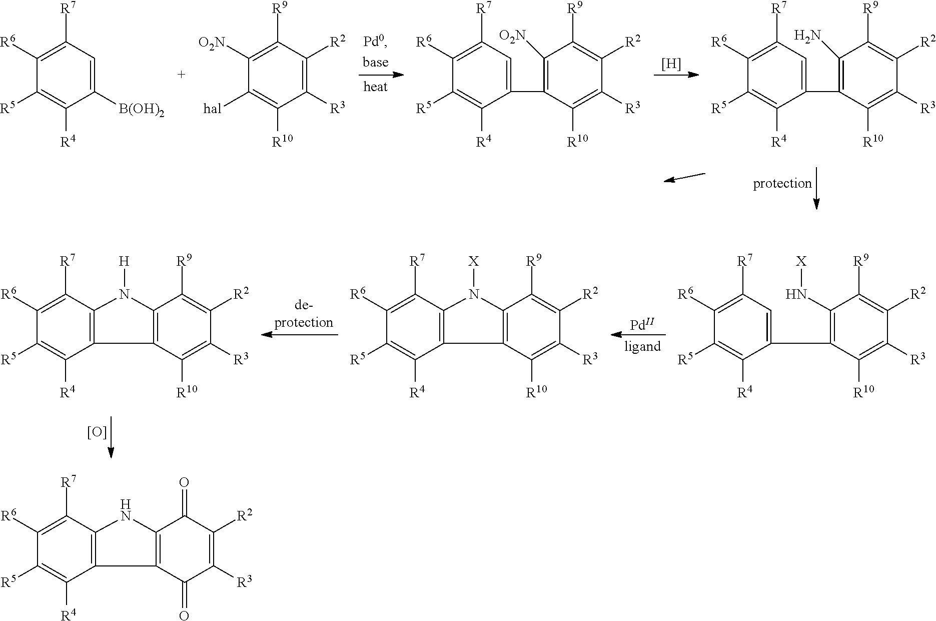

wherein R² to R⁷, R⁹ and R¹⁰ are as herein defined;
X denotes a suitable protecting group such as, but not limited to, acetyl (Ac), carbobenzyloxy (Cbz), tert-butyloxycarbonyl (Boc), 9-fluroneylmethyloxycarbonyl (Fmoc), benzoyl (Bz), benzyl (Bn), tosyl (Ts), trichloroethyl chloroformate (Troc), para-methoxybenzyl (PMB), 3,4-dimethoxybenzyl (DMPM), para-methoxyphenyl (PMP), and (o- or p-nitrobenzenesulfonyl (Nosyl); and
“hal” is a halogen atom, e.g. Cl, Br or I.

In this scheme, a Suzuki reaction is performed between the boronic acid and aryl halide starting materials to afford the bicyclic product. The nitro aromatic is then reduced to form the amino group, which can be protected with a standard protecting group (e.g. Ac). A ring closing step is then performed to afford the carbazole system. The protecting group(s) can then be removed. An oxidation step is performed to form the carbazole quinone, followed by a regioselective methylation. Alternatively, a direct cyclisation can be performed via intramolecular C—H activation and C—N bond formation.

Any of the compounds herein described for use in the invention may be converted into a salt thereof, particularly into a pharmaceutically acceptable salt thereof with an inorganic or organic acid or base. Acids which may be used for this purpose include hydrochloric acid, hydrobromic acid, sulfuric acid, sulfonic acid, methane sulfonic acid, phosphoric acid, fumaric acid, succinic acid, lactic acid, citric acid, tartaric acid, maleic acid, acetic acid, trifluoroacetic acid and ascorbic acid. Bases which may be suitable for this purpose include alkali and alkaline earth metal hydroxides, e.g. sodium hydroxide, potassium hydroxide or cesium hydroxide, ammonia and organic amines such as diethylamine, triethylamine, ethanolamine, diethanolamine, cyclohexylamine and dicyclohexylamine. Procedures for salt formation are conventional in the art.

The compounds described herein find use in the treatment or prevention of leukaemia in a subject or patient and, in particular, in the treatment or prevention of chronic myeloid leukaemia (CML), acute myeloid leukaemia (AML), acute lymphocytic leukaemia (ALL) and t-ALL (T-cell acute lymphoblastic leukaemia). For example, they may be used in the treatment of adult acute lymphoid leukaemia or Philadelphia chromosome positive acute lymphoid leukaemia (“Ph+ ALL”). As used herein, a “subject” or “patient” encompasses any animal, preferably a mammal. Examples of mammalian subjects include, without limitation, humans, dogs, cats, rodents (e.g. mouse, rat, guinea pig, etc.), horses, cattle, sheep, and pigs. Preferably, the subject is a human.

In one embodiment, the compounds herein described are suitable for preventing and/or retarding leukaemia cell proliferation, differentiation and/or survival, or for preventing and/or retarding metastasis of leukaemia cells. As used herein, the term “proliferation” refers to cells undergoing mitosis. The term “retarding proliferation” indicates that the compounds inhibit proliferation of a leukaemia cell. In preferred embodiments, “retarding proliferation” indicates that DNA replication is at least 10% less than that observed in untreated cells, more preferably at least 25% less, yet more preferably at least 50% less, e.g. 75%, 90% or 95% less than that observed in untreated cells.

In one aspect, the invention relates to a method of preventing or treating leukaemia in a subject (e.g. a human patient). This method involves selecting a subject having leukaemia or at risk of developing leukaemia and administering a compound as herein described to the selected subject under conditions effective to prevent or treat the leukaemia. In one embodiment, the subject has or is at risk of developing chronic myeloid leukaemia (CML), acute myeloid leukaemia (AML) or acute lymphocytic leukaemia (ALL).

In one embodiment, the compounds herein described may be used in the treatment of pre-malignant conditions, such as conditions in which pre-malignant precursors to leukaemia may be present. Such conditions include, for example, myelodysplastic syndrome which is a pre-malignant precursor to AML.

In one embodiment, suitable subjects for treatment in accordance with the invention are those subjects having leukaemia and that have developed resistance to conventional cancer therapies.

The compounds herein described can be administered alone in any of the methods herein described. Alternatively, these may be administered in combination with other known cancer therapies, including but not limited to, radiotherapy and hematopoietic stem cell transplantation, for example in consolidation therapy following stem cell transplantation therapy in leukaemia patients.

For use in a therapeutic or prophylactic treatment, the compounds herein described will typically be formulated as a pharmaceutical formulation. In a further aspect, the invention thus provides a pharmaceutical composition comprising a compound of formula (I) as herein described, together with one or more pharmaceutically acceptable carriers, excipients or diluents. Acceptable carriers, excipients and diluents for therapeutic use are well known in the art and can be selected with regard to the intended route of administration and standard pharmaceutical practice. Examples include binders, lubricants, suspending agents, coating agents, solubilizing agents, preserving agents, wetting agents, emulsifiers, surfactants, sweeteners, colorants, flavouring agents, antioxidants, odorants, buffers, stabilizing agents and/or salts.

The compounds for use in the invention may be formulated with one or more conventional carriers and/or excipients according to techniques well known in the art. Typically, the compositions will be adapted for oral or parenteral administration, for example by intradermal, subcutaneous, intraperitoneal, or intravenous injection. For example, these may be formulated in conventional oral administration forms, e.g. tablets, coated tablets, caplets, capsules, powders, granulates, solutions, dispersions, suspensions, syrups, emulsions, etc. using conventional excipients, e.g. solvents, diluents, binders, sweeteners, aromas, pH modifiers, viscosity modifiers, antioxidants, etc. Suitable excipients can readily be determined by those skilled in the art. The formulations may be prepared using conventional techniques, such as dissolution and/or mixing procedures.

Where parenteral administration is employed this may, for example, be by means of intravenous, subcutaneous, intraperitoneal or intramuscular injection. For this purpose, sterile solutions containing the active agent may be employed, such as an oil-in-water emulsion. Where water is present, an appropriate buffer system may be added to prevent pH drift under storage conditions.

The dosage required to achieve the desired activity of the compounds herein described will depend on various factors, such as the compound selected, its mode and frequency of administration, whether the treatment is therapeutic or prophylactic, and the nature and severity of the disease or condition, etc. Typically, a physician will determine the actual dosage which will be most suitable for an individual subject. The specific dose level and frequency of dosage for any particular patient may be varied and will depend upon factors such as the activity of the specific compound employed, the metabolic stability and length of action of that compound, the age of the patient, the mode and time of administration, and the severity of the particular condition. The compound and/or the pharmaceutical composition may be administered in accordance with a regimen from 1 to 10 times per day, such as once or twice per day. For oral and parenteral administration to human patients, the daily dosage level of the agent may be in single or divided doses.

Suitable daily dosages of the compounds herein described may readily be determined by those skilled in the art, but are expected to be in the range from 0.1 mg to 1 g of the compound; 1 mg to 500 mg of the compound; 1 mg to 300 mg of the compound; 5 mg to 100 mg of the compound, or 10 mg to 50 mg of the compound. By a “daily dosage” is meant the dosage per 24 hours.

### EXAMPLES

The invention will now be described in more detail in the following non-limiting Examples and with reference to the accompanying FIGURE, in which:

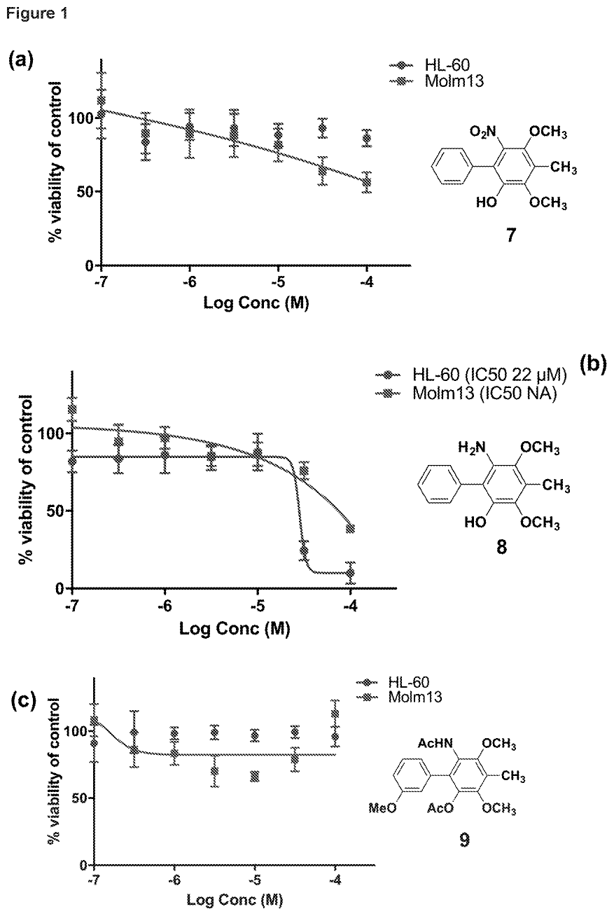

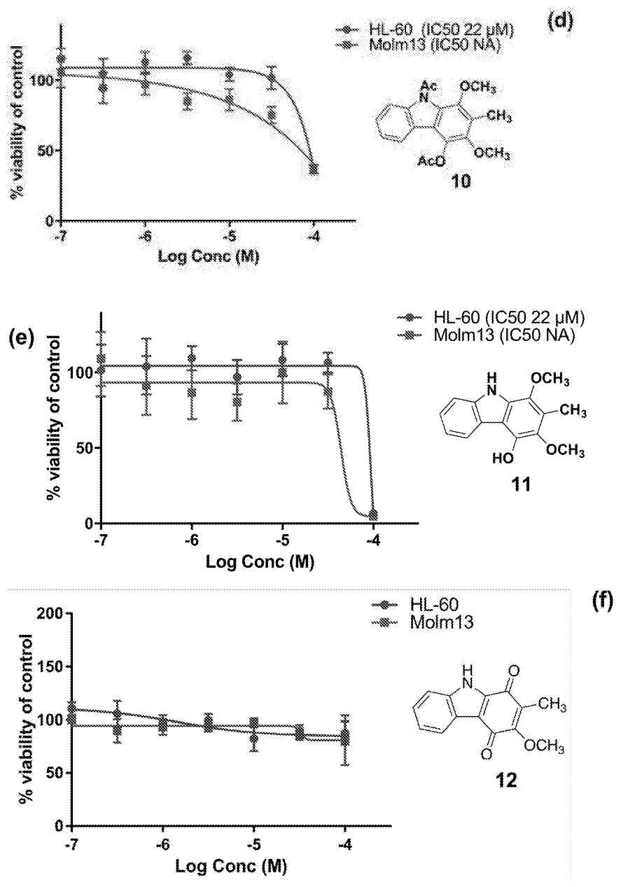

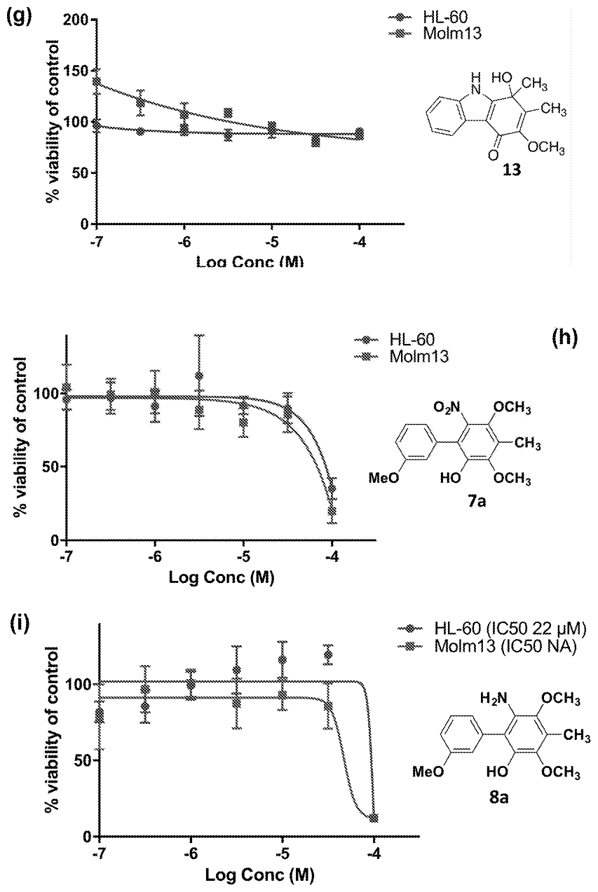

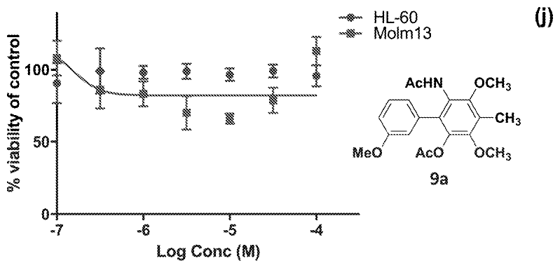

FIG. 1 shows the results from testing of compounds in the leukaemia cell lines HL60 and MOLM-13. The cell lines HL60 and MOLM-13 were treated with the various compounds for 24 hours. The cell viability assay WST-1 was used to determine the cytotoxic effect and to generate the dose-response curves and IC₅₀ values.

### Experimental Details

All reagents and solvents were purchased from commercial sources and used as received. Melting points were determined in open capillaries. Reagent grade chemicals were purchased from commercial sources and used without further purification. All reaction mixtures and column eluents were monitored by means of TLC (TLC plates Merck Kieselgel 60 F254). The TLC plates were observed under UV light at λ=254 nm and λ=365 nm. IR spectra were recorded as KBr discs with a Shimadzu FTIR-8300 spectrophotometer, and ¹H and ¹³C NMR spectra were recorded with the Brüker instruments AC-200F, DMX 400WB, DPX 400, AV 500, and Biospin 850SB. High-resolution mass spectra (HRMS) were performed with a Q-TOF Micro YA263 instrument.

### General Methods

GC analyses were performed with a capillary gas chromatograph equipped with a fused silica column (125 m, 0.20 mm i.d., 0.33 mm film thickness) at a helium pressure of 200 kPa, split less/split injector and flame ionization detector. Mass spectra were obtained with a GC-MS instrument, with a gas chromatograph equipped with a fused silica column (130 m, 0.25 mm i.d., 0.25 mm film thickness) and helium as the carrier gas. DART mass spectra were obtained by using PEG as an internal standard in positive ionization mode with a TOF mass analyzer. ¹H and ¹³C NMR spectra were recorded at ambient temperature at a frequency of 400, 500, and 850 MHz and 100, 125, 212.5 MHz respectively. The chemical shifts are reported in ppm relative to residual CDCl₃ for proton (δ=7.26 ppm), CDCl₃ for carbon (δ=77.0 ppm), DMSO-da for proton (δ=2.50 ppm), and DMSO-de for carbon (δ=39.51 ppm) with tetramethylsilane as an external reference. Flash chromatography was performed by using the indicated solvent system and silica gel (230-400 mesh). All reagents used were commercially available from Aldrich Chemical Co. The microwave-assisted experiments were performed by means of a Biotage Initiator Sixty EXP Microwave System, that operates at 0-400 W at 2.45 GHz, in the temperature range of 40-250° C., a pressure range of 0-20 bar (2 MPa, 290 psi), and with reactor vial volumes of 0.2-20 mL.

### Example 1—Synthesis of Compounds

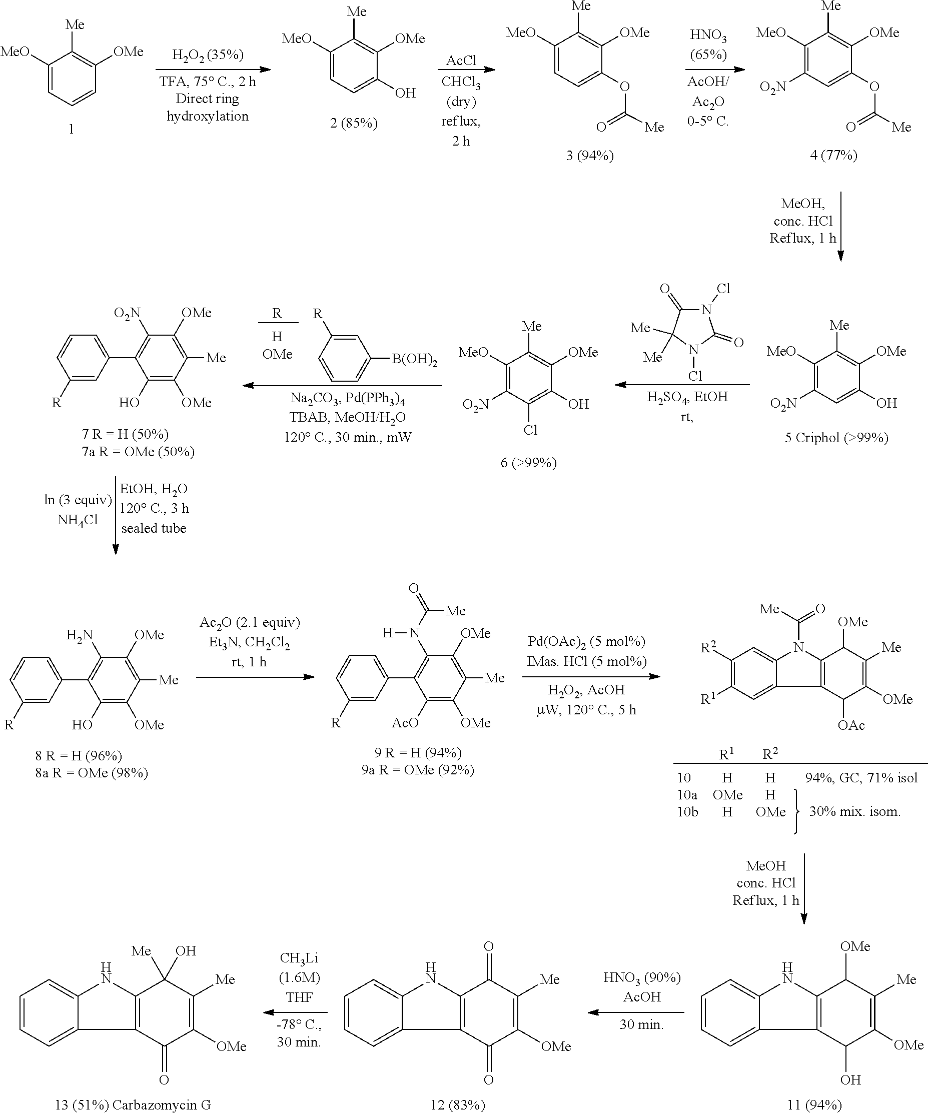

### Preparation of 2,4-dimethoxy-3-methyl phenol (2)

To a solution of 2,6-dimethoxy toluene 1 (3 g, 19.7 mmol) in acetonitrile (25 mL) was added trifluoroacetic acid (1.3 mL, 19.7 mmol) and hydrogen peroxide (35%, 3.5 mL, 39.4 mmol). The reaction mixture was stirred for 2 h at 75° C. The reaction mixture was cooled to room temperature and the solvent acetonitrile was removed under reduced pressure. The crude mixture was diluted with water (30 mL) and extracted with EtOAc (2×40 mL). The organic layers were combined and dried over Na₂SO₄. The solvent was evaporated under reduced pressure. The title compound was isolated by silica gel column chromatography [(EtOAc:Hx, 20:80)] as an orange liquid (2.85 g, 85%).

R_{f}=0.49 [(EtOAc:Hx, 20:80)].

¹H-NMR (500 MHz, CDCl₃): δ=6.67 (d, J=9 Hz, 1H), 6.46 (d, J=9 Hz, 1H), 5.10 (s, br, 1H), 3.69 (s, 6H), 2.09 (s, 3H).

¹³C-NMR (125 MHz, CDCl₃,): δ=151.9, 145.9, 142.8, 120.1, 111.8, 106.9, 60.9, 56.1, 9.3; MS (EI): m/z (%); 168 (100, M⁺), 153 (61), 125 (49), 107 (23), 93 (9), 79 (11), 65 (21), 53 (12); IR (cm⁻¹): 3406, 2996, 2940, 2834, 1486, 1259, 1098, 730.

### Preparation of 2,4-dimethoxy-3-methylphenyl acetate (3)

To a solution of 2,4-dimethoxy-3-methyl phenol 2 (1.5 g, 8.9 mmol) in CHCl₃ (15 mL), was added acetyl chloride (1.3 mL, 17.8 mmol). The reaction mixture was heated for 2 h under reflux. The reaction mixture was cooled to ambient temperature and the solvent was removed under reduced pressure to afford the title compound as a dark orange liquid (1.75 g, 94%).

R_{f}=0.78 [(EtOAc:Hx, 40:60)].

¹H-NMR (500 MHz, CDCl₃): δ=6.86 (d, J=8.5 Hz, 1H), 6.60 (d, J=8.5 Hz, 1H), 3.80 (s, 3H), 3.75 (s, 3H), 2.32 (s, 3H), 2.17 (s, 3H).

¹³C-NMR (125 MHz, CDCl₃): δ=169.7, 156.4, 150.5, 137.6, 121.2, 119.7, 105.6, 60.8, 55.7, 20.7, 9.2; MS (EI): m/z (%): 210 (9, M⁺), 168 (100), 153 (53), 139 (4), 125 (19), 107 (12), 79 (6), 65 (8), 53 (7); IR (cm⁻¹): 2940, 2838, 1759, 1484, 1199, 1106, 728.

### Preparation of 2,4-dimethoxy-3-methyl-5-nitrophenyl acetate (4)

2,4-Dimethoxy-3-methylphenyl acetate 3 (1.6 g, 7.6 mmol) was dissolved in a solution of AcOH/Ac₂O (1:3 ratio, 12 mL) at 0-5° C. A solution of HNO₃ (65%, 1.0 mL, 14.5 mmol) in AcOH/Ac₂O (1:3 ratio, 12 mL) was added dropwise to the reaction mixture under vigorous stirring at 0-5° C. When the addition was completed, the reaction mixture was stirred for 30 minutes at ambient temperature. The reaction mixture was poured into water (100 mL) and extracted with CH₂Cl₂ (3×30 mL). The organic layers were combined and washed with NaHCO₃ (2×50 mL) and dried over Na₂SO₄. The solvent was evaporated under reduced pressure to afford the title compound as a yellow liquid (1.49 g, 77%).

R¹=0.70 [(EtOAc:Hx, 40:60)].

¹H-NMR (500 MHz, CDCl₃): δ=7.54 (s, 1H), 3.87 (s, 3H), 3.83 (s, 3H), 2.33 (s, 3H), 2.26 (s, 3H).

¹³C-NMR (125 MHz, CDCl₃): δ=168.7, 155.2, 151.3, 139.1, 138.8, 129, 118, 62.1, 60.9, 20.6, 10; MS (EI): m/z (%): 255 (4, M⁺), 213 (100), 166 (26), 152 (8), 137 (8), 123 (11), 107 (10), 77 (14), 53 (14); IR (cm): 2946, 1770, 1522, 1343, 1187, 988, 729.

### Preparation of 2,4-dimethoxy-3-methyl-5-nitrophenol (5)

A solution of conc. HCl (12 mL) in MeOH (25 mL) was added dropwise to 2,4-dimethoxy-3-methyl-5-nitrophenyl acetate 4 (1.5 g, 5.9 mmol) at 0° C. The reaction mixture was stirred for 1 h at 70° C. under reflux. The reaction mixture was cooled to ambient temperature and the solvent was removed under reduced pressure. The crude product was diluted with water (40 mL) and extracted with EtOAc (2×40 mL). The organic layers were combined and dried over Na₂SO₄ to afford the Criphol 5 as an orange colored solid (1.25 g, 99%).

R_{f}=0.34 [(EtOAc:Hx, 20:80)]; mp 53-55° C.

¹H NMR (500 MHz, CDCl₃): δ=7.26 (s, 1H), 3.78 (s, 6H), 2.21 (s, 3H).

¹³CNMR (125 MHz, CDCl₃): δ=150.2, 146.3, 145.1, 140.0, 127.5, 109.4, 62.1, 61.0, 10; MS (EI): m/z (%): 213 (100, M⁺), 166 (59), 152 (21), 137 (36), 125 (43), 122 (28), 91(23), 83(40), 77 (32), 53 (49); IR (cm⁻¹): 3418, 2944, 1519, 1338, 1245, 1102, 988, 733.

### Preparation of 2-chloro-4,6-dimethoxy-5-methyl-3-nitrophenol (6)

To a solution of 2,4-dimethoxy-3-methyl-5-nitrophenol 5 (0.311 g, 0.145 mmol) in EtOH (15 mL), DCH (146 mg, 0.074 mmol) was added followed by drop-wise addition of conc. H₂SO₄ (≈24 drops) under good stirring. After the addition was completed, the reaction mixture was quenched with NaOH (4.1 M, 5 mL). A heavy red precipitation was observed during the addition of NaOH, which was neutralized with acetic acid (pH≈4), and the resulting mixture was diluted with water (25 mL) and extracted with diethyl ether (3×40 mL). The organic layers were combined and dried with Na₂SO₄. The crude product was isolated by silica gel column chromatography (CH₂Cl₂/hexane, 40:60) to afford the title compound as pale yellow crystals (0.34 g, 95%), m.p. 118.5° C.

R^{f}=0.29 (CH₂Cl₂/hexane, 60:40).

¹H-NMR (400 MHz, CDCl₃): δ=5.91 (s, 1H), 3.87 (s, 3H), 3.80 (s, 3H), 2.26 (s, 3H) ppm.

¹³C-NMR (100 MHz, CDCl₃): δ=147.5, 144.0, 142.7, 125.6, 109.3, 62.9, 61.0, 9.9 ppm. MS (EI): m/z (%)=247 (100, M⁺), 217 (5), 213 (16), 186 (25), 171 (27), 138 (26), 108 (20), 83 (22), 77 (48), 67 (32).

HRMS (EI): Calcd. for C₉H₁₀ClNO₅ 247.0248. Found 247.0248. IR (cm⁻¹)=3406, 3083, 2970, 2929, 2846, 1500, 1108.

### Preparation of 3,5-dimethoxy-4-methyl-6-nitrobiphenyl-2-ol (7)

In a microwave tube, 2-chloro 4,6-dimethoxy-5-methyl-3-nitrophenol 6 (0.21 g, 0.85 mmol), phenylboronic acid (0.155 g, 1.27 mmol), Na₂CO₃ (0.10 g, 0.97 mmol), TBAB (0.021 g, 0.065 mmol) and Pd(PPh₃)₄ (0.025 g, 0.022 mmol) were added. The reaction mixture was carefully flushed with argon before adding a mixture of MeOH (4 mL) and water (1 mL). The tube was sealed and placed in the microwave cavity for 30 min. at 120° C. The reaction mixture was diluted with water (40 mL) and extracted with diethyl ether (2×30 mL). The organic layers were combined and dried over Na₂SO₄. The crude product was isolated by silica gel column chromatography [(DCM:Hx, 40:60)] eluent to afford the title compound as a yellow solid (0.122 g, 50%); R_{f}=0.1 (CH₂Cl₂/hexane, 40:60); mp: 102.7° C.

¹H-NMR (500 MHz, CDCl₃): δ=7.46-7.40 (m, 3H), 7.37-7.35 (m, 2H), 5.55 (s, 1H), 3.88 (s, 3H), 3.84 (s, 3H), 2.33 (s, 3H).

¹³C-NMR (125 MHz, CDCl₃): δ=147.2, 143.2, 142.9, 142.6, 130.8, 129.4, 128.9, 128.8, 125.6, 119.4, 62.8, 61.0, 10; MS (EI): m/z (%): 289 (100, M⁺), 272 (7), 246 (20), 207 (24), 199 (13), 169 (13), 141 (20), 129 (40), 115 (33), 102 (12), 83 (23), 77 (20).

HR-MS (EI): (M+Na)⁺: Calcd for C₁₅H₁₅NNaO₅ 312.0848. Found 312.0846);: IR (cm⁻¹)=3413, 2937, 2929, 2849, 1528, 1101, 991, 748.

### Preparation of 3,3′,5-trimethoxy-4-methyl-6-nitro-[1,1′-biphenyl]-2-ol (7a)

In a microwave reactor tube, 2-chloro-4,6-dimethoxy-5-methyl-3-nitrophenol 6 (0.168 g, 0.68 mmol), 3-methoxy phenylboronic acid (0.155 g, 1.02 mmol), Na₂CO₃ (0.079 g, 0.75 mmol), TBAB (0.019 g, 0.05 mmol) and Pd(PPh₃)₄ (0.020 g, 0.018 mmol) were added. The reaction mixture was carefully flushed with argon before adding a mixture of MeOH (4 mL) and water (1 mL). The tube was sealed and submerged in the microwave cavity for 30 min at 120° C. The reaction mixture was diluted with water (40 mL) and extracted with diethyl ether (2×30 mL). The organic layers were combined and dried over Na₂SO₄. The crude product was isolated by silica gel column chromatography [(DCM:Hx, 40:60)] eluent to afford the title compound as a yellow oil (0.109 g, 50%); R_{f}=0.1 (CH₂Cl₂/hexane, 40:60).

¹H-NMR (500 MHz, CDCl₃): δ=7.28 (t, J=8 Hz, 1H), 6.88-6.85 (m, 2H), 6.82 (s, 1H), 5.48 (s, 1H), 3.80 (s, 3H), 3.76 (s, 3H), 3.73 (s, 3H), 2.25 (s, 3H).

¹³C-NMR (125 MHz, CDCl₃): δ=159.8, 147.2, 143.2, 142.8, 142.4, 131.9, 130, 125.7, 121.6, 119.2, 114.9, 114.8, 62.8, 60.9, 10.

MS (EI): m/z (%): 320 (17), 319 (100, M⁺), 276 (33), 257 (15), 244 (22), 159 (32), 128 (35), 115 (45), 83 (38), 55 (22).

HR-MS (DART): (M+H)⁺: Calcd for C₁₈H₁₈NO₆ 320.1134. Found 320.1136); IR (cm⁻¹)=3450, 2942, 2837, 1530, 1238, 1103, 997, 765.

### Preparation of 6-amino-3,5-dimethoxy-4-methyl-[1,1′-biphenyl]-2-ol (8)

3,5-Dimethoxy-4-methyl-6-nitro-[1,1′-biphenyl]-2-ol 7 (0.20 g, 0.69 mmol) was dissolved in EtOH (4 mL) and transferred to a tube reactor. Then, a mixture of NH₄Cl (0.073 g, 1.38 mmol) in H₂O (1.2 mL) and indium powder (0.238 g, 2.01 mmol) were added whereupon a magnetic stirrer bar was transferred to the tube. The tube was then sealed and the reaction mixture was stirred and heated at 120° C. for 3 h. The reaction mixture was cooled to room temperature and diluted with ethyl acetate (30 mL) and filtered through a pad of celite. Another portion (20 mL) of ethyl acetate was used to wash through the filter pad. The resulting organic phase was dried over Na₂SO₄, filtered and the solvent was removed under reduced pressure to obtain compound 8 as a purple oil (0.175 g, 98%); R_{f}=0.44 [(EtOAc:Hx, 30:70)].

¹H-NMR (500 MHz, CDCl₃): δ=7.50-7.47 (m, 3H), 7.43-7.40 (m, 2H), 5.30 (s, 1H), 3.76 (s, 3H), 3.74 (s, 3H), 2.27 (s, 3H).

¹³C-NMR (125 MHz, CDCl₃): δ=142.8, 138.5, 137.2, 134.4, 134.0, 130.5, 129.1, 127.7, 123.3, 113.0, 61.1, 59.5, 9.5.

MS (EI): m/z (%): 260 (9), 259 (58, M⁺), 244 (100), 229 (9), 216 (22), 201 (36), 184 (11), 144 (14), 128(11), 89 (8), 77(9).

HR-MS (DART): (M+H)⁺: Calcd for C₁₅H₁₈NO₃ 260.1287. Found 260.1287); IR (cm⁻¹): 3458, 3334, 3057, 2933, 2849, 1580, 1460, 994, 727.

### Preparation of 6-amino-3,3′,5-trimethoxy-4-methyl-[1,1′-biphenyl]-2-ol (8a)

3,3′,5-trimethoxy-4-methyl-6-nitro-(1,1′-biphenyl)-2-ol (7a) (0.08 g, 0.25 mmol) was dissolved in EtOH (4 mL) and transferred to a tube reactor. Then, a slurry of NH₄Cl (0.027 g, 0.50 mmol) in H₂O (1.2 mL) and indium powder (0.086 g, 0.75 mmol, 99.99% 100 mesh) were added whereupon a magnetic stirrer bar was transferred to the tube. The tube was then sealed and the reaction mixture was stirred and heated at 120° C. for 3 h. The reaction mixture was cooled to room temperature and diluted with ethyl acetate (20 mL) and filtered through a pad of celite. Another portion (20 mL) of ethyl acetate was used to wash through the filter pad. The resulting organic phase was dried over Na₂SO₄, filtered and the solvent was removed under reduced pressure using a rotary evaporator to obtain the compound 8a in purple oil (0.071 g, 98%); R_{f}=0.36 [(EtOAc:Hx, 30:70)].

¹H-NMR (500 MHz, CDCl₃): δ=7.40 (t, J=8 Hz, 1H), 6.99-6.97 (m, 1H), 6.95-6.94 (m, 1H), 6.93-6.91 (m, 1H), 5.29 (s, 1H), 3.82 (s, 3H), 3.76 (s, 3H), 3.74 (s, 3H), 2.27 (s, 3H).

¹³C-NMR (125 MHz, CDCl₃): δ=160.1, 142.8, 138.5, 137.1, 135.3, 134.4, 130.2, 123.4, 122.7, 115.8, 113.6, 112.8, 61.0, 59.5, 55.3, 9.5.

MS (EI): m/z (%): 290 (12), 289 (74, M⁺), 274 (100), 259 (12), 246 (26), 231 (25), 137 (14), 130 (32), 115 (15), 83 (14), 77(10).

HR-MS (DART): (M+H)⁺: Calcd for C₁₆H₂₀NO₄ 290.1392. Found 290.1395); IR (cm¹): 3458, 3341, 3057, 2933, 2849, 1580, 1360, 994, 727.

### Preparation of 6-acetamido-3,5-dimethoxy-4-methyl-(1,1′-biphenyl)-2-yl acetate (9)

In a round bottom flask (50 mL), 6-amino-3,5-dimethoxy-4-methyl-(1,1′-biphenyl)-2-ol 8 (0.12 g, 0.46 mmol) was dissolved in dry CH₂Cl₂ (10 mL) under inert atmosphere. To the stirred solution added triethylamine (0.14 mL, 0.97 mmol). The solution was cooled under ice bath and added acetic anhydride (0.09 mL, 1.01 mmol) dropwise to the reaction mixture. The reaction mixture was stirred at room temperature for 1 hour. The residue was diluted in EtOAc (50 mL) and washed with water (40 mL). The organic layer was washed with NaHCO₃ (30 mL) and finally dried over Na₂SO₄. The solvent was evaporated under reduced pressure to obtain the acetylated product 9 (0.148 g, purple liquid) in 94% crude yield.

R_{f}=0.74 [(EtOAc:Hx, 50:50)].

¹H-NMR (500 MHz, CDCl₃): δ=7.34 (m, 3H), 7.20 (m, 2H), 6.50 (s, br, 1H), 3.76 (d, J=8.5 Hz, 6H), 2.29 (s, 3H), 2.17 (s, 3H), 1.99 (s, 3H).

MS (EI): m/z (%): 343 (11, M⁺), 301 (46), 270 (24), 244 (100), 201 (24), 144 (51), 128 (39), 115 (60), 89 (72), 83(94), 77(47), 55(43).

HR-MS (DART): (M+H)⁺: Calcd for C₁₉H₂₂NO₅ 344.14980. Found 344.14999; IR (cm⁻¹): 2928, 2552, 1669, 1603, 1451, 1422, 1290, 1205, 707.

### Preparation of 6-acetamido-3,3′,5-trimethoxy-4-methyl-(1,1′-biphenyl)-2-yl acetate (9a)

In a round bottom flask (50 mL), 6-amino-3,3′,5-trimethoxy-4-methyl-(1,1′-biphenyl)-2-ol 8a (0.13 g, 0.45 mmol) was dissolved in dry CH₂Cl₂ (10 mL) under inert atmosphere. Triethylamine (0.13 mL, 0.97 mmol) was then added to the stirred mixture. The solution was cooled under ice bath and added acetic anhydride (0.09 mL, 1.01 mmol) drop-wise to the reaction mixture. The reaction mixture was stirred at room temperature for 1 hour. The residue was diluted in EtOAc (50 mL) and washed with water (40 mL). The organic layer was washed with NaHCO₃ (30 mL) and finally dried over Na₂SO₄. The solvent was evaporated under reduced pressure to obtain the acetylated product 9a (0.131 g, purple liquid) in 92% crude yield. The acetylated product 9a was used in the next step without further purification.

R_{f}=0.67 [(EtOAc:Hx, 50:50)].

MS (EI): m/z (%): 373 (16, M⁺), 332 (12), 331 (68), 316(7), 300 (26), 274 (100), 256 (11), 242 (13), 231 (11), 174 (12), 115 (9), 83 (15).

### Preparation of 9-acetyl-1,3-dimethoxy-2-methyl-9H-carbazol-4-yl acetate (10)

6-Acetamido-3,5-dimethoxy-4-methyl-(1,1′-biphenyl)-2-yl acetate 9 (0.063 g, 0.183 mmol) was dissolved in glacial acetic acid (5 mL), Pd(OAc)₂ (2.1 mg, 0.009 mmol), IMes·HCl (3.2 mg, 0.009 mmol), and H₂O₂ (35%, 0.05 mL, 0.53 mmol) were added. The vial was sealed whereupon a magnetic stirrer bar was transferred to the tube. The tube was submerged in the microwave cavity at 120° C. for 5 h. The reaction mixture was monitored by means of GC (94% yield). The crude product was dissolved in EtOAc (20 mL) and washed with water (25 mL). The water phase was extracted with EtOAc (2×15 mL). The combined layer was washed with aq. NaHCO₃ (20 mL). The organic layer was dried over Na₂SO₄ and filtered off. The solvent was evaporated under reduced pressure. The crude product was purified by using silica gel column chromatography (20:80, EtOAc:Hx) to obtain the N-acetyl Carbazole compound 10 (0.043 g, brown liquid) in 71% yield.

R_{f}=0.33 [(EtOAc:Hx, 20:80)].

¹H-NMR (500 MHz, CDCl₃): δ=8.26 (d, J=8.5 Hz, 1H), 7.83 (d, J=7.5 Hz, 1H), 7.45 (t, J=7.5 Hz, 1H), 7.33 (t, J=7.5 Hz, 1H), 3.85 (s, 3H), 3.73 (s, 3H), 2.62 (s, 3H), 2.54 (s, 3H), 2.39 (s, 3H).

¹³C-NMR (125 MHz, CDCl₃): δ=172.8, 168.6, 147.4, 144.0, 140.4, 134.8, 128.5, 127.7, 124.9, 123.7, 123.6, 121.1, 119.7, 115.1, 61.1, 60.0, 26.8, 20.8, 10.2.

MS (EI): m/z (%): 341 (11, M⁺), 299 (19), 257 (46), 242 (100), 226 (11), 196 (12), 168 (22), 154 (23), 127 (22), 115 (27), 89(12), 77(12), 55(12).

HR-MS (DART): (M+H)⁺: Calcd for C₁₉H₂₀NO₅ 342.1341. Found 342.1343); IR (cm⁻¹): 2935, 1766, 1702, 1445, 1398, 1367, 1269, 1254, 1183, 1082, 1002, 749.

### Preparation of 9-acetyl-1,3,6-trimethoxy-2-methyl-9H-carbazol-4-yl acetate (10a) & 9-acetyl-1,3,8-trimethoxy-2-methyl-9H-carbazol-4-yl acetate (10b) (mixture of isomers)

6-Acetamido-3,3′,5-trimethoxy-4-methyl-[1,1′-biphenyl]-2-yl acetate 9a (0.130 g, 0.348 mmol) was dissolved in glacial acetic acid (5 mL), Pd(OAc)₂ (8 mg, 0.024 mmol), IMes·HCl (12 mg, 0.024 mmol) and H₂O₂ (35%, 0.09 mL, 1.0 mmol) were added. The vial was sealed whereupon a magnetic stirrer bar was transferred to the tube. The tube was submerged in the microwave cavity at 120° C. for 5 hours. The reaction mixture was monitored by means of GC (50% yield). Acetic acid was removed under reduced pressure. The crude product was dissolved in EtOAc (40 mL) and washed with water (25 mL). The water phase was extracted with EtOAc (2×40 mL). The combined organic layer was washed with aq. NaHCO₃ (30 mL). The organic layer was dried over Na₂SO₄ and filtered off. The solvent was evaporated under reduced pressure. The crude product was purified by using silica gel column chromatography (20:80, EtOAc:Hx) which afforded a mixture of the isomers 10a and 10b in a yield of 30%.

¹H-NMR (500 MHz, CDCl₃): δ=8.13 (d, J=9 Hz, 1H), 7.64 (d, J=7.5 Hz, 1H), 7.54-7.55 (m, 1H), 7.32 (t, J=8 Hz, 1H), 7.25 (m, 1H), 6.96-6.98 (dd, J=2.5, 9 Hz, 1H), 3.82 (s, 1H), 3.79 (s, 3H), 3.77 (s, 1H), 3.65 (s, 3H), 2.54 (s, 3H), 2.47 (s, 3H), 2.31 (s, 3H).

¹³C-NMR (125 MHz, CDCl₃): δ=172.5, 168.6, 159.6, 156.3, 147.4, 144.2, 135.1, 134.7, 130.5, 129.5, 129.1, 125.1, 124.6, 122.6, 120.4, 119.7, 116.2, 114.4, 114.3, 105.5, 61.1, 59.9, 55.8, 55.5, 26.6, 20.8, 10.2.

MS (EI): m/z (%): 371 (7, M⁺), 331 (11), 287 (28), 272 (62), 207 (69), 115 (34), 96 (38), 83 (42), 77(76).

### Preparation of 1,3-dimethoxy-2-methyl-9H-carbazol-4-ol (11)

To a stirred solution of 9-acetyl-1,3-dimethoxy-2-methyl-9H-carbazol-4-yl acetate 10 (0.023 g, 0.07 mmol) in methanol (10 mL) at 0° C. was added a solution of conc. HCl (2 mL) in MeOH (5 mL) dropwise. The reaction mixture was stirred for 1 h at 70° C. The reaction mixture was cooled to room temperature. The reaction mixture was diluted with water (40 mL) and extracted with EtOAc (2×20 mL). The combined organic layer was dried over Na₂SO₄. The solvent was filtered off to obtain the compound 11 as a brown solid (0.017 g, 94%).

R_{f}=0.42[(EtOAc:Hx, 20:80)].

¹H-NMR (500 MHz, CDCl₃): δ=8.24 (d, J=8 Hz, 1H), 8.10 (s, br, 1H), 7.41 (m, 1H), 7.39-7.35 (td, J=1.0 Hz, 6.5 Hz, 1H), 7.24-7.20 (m, 1H), 6.03 (s, 1H), 3.90 (s, 3H), 3.85 (s, 3H), 2.42 (s, 3H).

¹³C-NMR (125 MHz, CDCl₃): δ=140.7, 139.1, 137.6, 135.8, 130.7, 125.0, 123.2, 122.6, 121.0, 119.5, 110.6, 110.2, 61.3, 60.8, 9.8.

HR-MS (DART): (M+H)⁺: Calcd for C₁₅H₁₆NO₃ 258.1130. Found 258.1133); IR (cm⁻¹): 3309, 3244, 2973, 2924, 2896, 1624, 1321, 1087, 1046, 879.

### Preparation of 3-methoxy-2-methyl-1-carbazole-1,4 (9H)-dione (12)

To a solution of 1,3-dimethoxy-2-methyl-9H-carbazol-4-ol (11) (40 mg, 0.155 mmol) in glacial acetic acid (3 mL), HNO₃ (90%, 0.01 mL, 0.186 mmol) was added slowly at 0-5° C. Then, the reaction mixture was stirred at ambient temperature for 15 min. After the reaction time, H₂O (20 mL) was added to the solution and extracted with EtOAc (3×30 mL). The combined organic extracts were washed with H₂O (2×40 mL) and dried over MgSO₄ and filtered. The solvent was evaporated under reduced pressure. The crude product was purified by using silica gel column chromatography (50:50, DCM:Hx) to obtain the compound 12 as a pale-green solid (36 mg, 0.124 mmol).

R_{f}=0.86[(EtOAc:Hx, 50:50)].

¹H-NMR (500 MHz, DMSO-de): 6=12.85 (s, br, 1H), 8.01 (d, J=8 Hz, 1H), 7.53 (m, 1H), 7.35-7.38 (td, J=1.0, 8.0 Hz, 1H), 7.29-7.32 (td, J=1.0 Hz, 8.0 Hz, 1H), 4.03 (s, 3H), 1.92 (s, 3H).

¹³C-NMR (125 MHz, DMSO-d₆): δ=180.5, 178.7, 157.8, 137.6, 136.4, 129.3, 128.4, 126.6, 126.0, 123.8, 121.4, 113.7, 61.2, 8.4.

HR-MS (ESI−): (M−H)⁺: Calcd for C₁₄H₁₀NO₃ 240.06606. Found 240.06632); IR (cm⁻¹): 3257, 2958, 2923, 2853, 1639, 1258, 1094, 1022, 794.

### Preparation of Carbazomycin G (1-hydroxy-3-methoxy-1,2-dimethyl-1,9-dihydro-4H-carbazol-4-one) (13)

To a vacuum dried Schlenk tube, the solution of compound 12 (0.022 g, 0.091 mmol) dissolved in THF (10 mL) was added under Ar atmosphere. The solution was cooled at −7° C. (using a dry ice and acetone mixture). A solution of methyllithium (1.6 M in Et₂O, 0.26 mL, 0.42 mmol) was added dropwise at −78° C. The reaction mixture was left (≈30 min.) to approach room temperature. Then NH₄Cl (10%, 10 mL) was added to quench the reaction whereupon the post-reaction mixture was extracted with EtOAc (2×50 mL). The organic extracts were combined and dried over Na₂SO₄. The solvent was removed under reduced pressure. The crude product was purified by using silica gel column chromatography (50:50, EtOAc:Hx) to obtain the compound 13 as a pale-yellow solid (51%, 12 mg, 0.047 mmol).

R_{f}=0.41[(EtOAc:Hx, 50:50)].

¹H-NMR (850 MHz, DMSO-d₆): δ=12.19 (s, br, 1H), 8.01 (d, J=7.7 Hz, 1H), 7.44 (d, J=7.7 Hz, 1H), 7.22 (t, J=7.7 Hz, 1H), 7.17 (t, J=7.7 Hz, 1H), 5.92 (s, 1H), 3.69 (s, 3H), 1.98 (s, 3H), 1.57 (s, 3H).

¹³C-NMR and DEPT (212.5 MHz, DMSO-d₆): δ=177.6 (C═O), 154.4 (C), 147.7 (C), 140.8 (C), 136.5 (C), 123.9 (C), 122.9 (CH), 121.4 (CH), 120.5 (CH), 112.0 (CH), 108.4 (C), 67.4 (C), 59.2 (CH₃), 27.9 (CH₃), 10.1 (CH₃).

HR-MS (ESI−): (M−H)⁺: Calcd for C₁₅H₁₄NO₃ 256.09737. Found 256.09799); IR (cm⁻¹): 3255 br, 2924, 2853, 1719, 1643, 1618, 1468, 1375, 1289, 1138, 1092, 1011, 961, 804, 748.

### Example 2—Testing of Compounds

The two human cell lines HL60 and MOLM-13 reflect the aggressive blood cancer acute myeloid leukaemia (AML). These cell lines (obtained from the American Type Culture Collection, ATCC) were cultured in RPMI 1640, 2 mM L-Glutamine, 50 UmL⁻¹ penicillin/streptomycin (Sigma Aldrich) and 10% fetal bovine serum (Biowest) and incubated in humidified atmosphere at a temperature of 37° C. under 5% CO₂. The cells (2×10⁵ cells×mL⁻¹) were treated with various concentrations (0.001 μM→100 μM) of the compounds for a period of 24 hours. Metabolic cell activity (i.e. cell viability) was measured using the WST-1 cell proliferation agent (Roche) and read on a luminescence plate reader (Infinite 200 Pro, Tecan). Selected cell samples were analysed after nuclear staining with Hoechst to determine apoptotic nuclear fragmentation.

The results are presented in FIG. 1 for the compounds 7-13. In general, all compounds were found to have the same effect on the two cell lines, and apparently the genotype or phenotype of these two specific cell lines had no immediate impact on response. Compounds 8, 10, 11, 7a, and 8a demonstrated a rapid decrease in cell survival in the mitochondria metabolism assay (WST-1). Though not wishing to be bound by theory, the cooperativity in cell death observed may reflect an enzymatic program or signaling cascade that destroys the cell. The compounds with more limited dose/response curves (7, 9, 9a, 12, and 13) may kill cells by a more passive interaction with the cell's life support systems.

Interestingly, a minor decrease in %-viability was observed when the two cell lines HL60 and MOLM-13 were treated with the bi-phenyl compound 7. Chemical reduction of the nitro group of compound 7 into the corresponding amino derivative 8 caused a substantial drop in %-viability, especially for the cell line HL60. This suggests that the amino group could be favourable for the observed cytotoxic activity. In the following step of the synthesis, the amino and hydroxyl groups of compound 8 were acetylated (protected). The obtained 6-acetamido-[1,1′-biphenyl]-2-yl acetate 9 displayed weaker cytotoxicity versus both of the two cell lines. It appears that the amino group of the molecular scaffold 8 is thus favourable for the cytotoxicity. The ring closure step of the synthesis that generates the carbazole scaffold 10 did not show any influence on the cytotoxicity. However, when the protective acetyl groups were removed in the deprotection step which affords the carbazole derivative 11, a major cytotoxicity towards both of the two cell lines was seen. Compound 11 is the result of rigidification of the structure that is compound 8. This knowledge taken together with the result of taking away ring aromaticity when the benzene ring of compound 11 is oxidised to compound 12 suggests that the periplanar form may be more beneficial.

1. A compound of formula (I), a stereoisomer, or a pharmaceutically acceptable salt thereof, for use in the treatment or prevention of leukaemia: 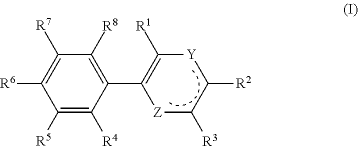
wherein:
Y is selected from C═O and —CR⁹ (in which R⁹ is H, —OH or —O-alkyl, (e.g. —O—C₁₋₆ alkyl));
Z is selected from C═O and —CR¹⁰ (in which R¹⁰ is H, —OH, —O-alkyl, (e.g. —O—C₁₋₆ alkyl) or —O—C(O)R^{A} wherein R^{A} is H or alkyl (e.g. C₁₋₆ alkyl));
R¹ is —NO₂ or —NR¹¹R¹² (wherein R¹¹ and R¹² are both H, or R¹¹ is H and R¹² is a group of the formula —C(O)R^{A} in which R^{A} is H or alkyl (e.g. C₁₋₆ alkyl));
each of R², R⁴, and R⁶ to R⁸ are independently selected from H, alkyl (e.g. C₁₋₆ alkyl), —O-alkyl (e.g. —O—C₁₋₆ alkyl), and halogen;
R³ and R⁵ are independently selected from H, —O-alkyl (e.g. —O—C₁₋₆ alkyl), and halogen;
or R¹ and R⁸ together form a group NR¹³ (in which R¹³ is H, alkyl (e.g. C₁₋₈ alkyl), or a group of the formula —C(O)R^{A} in which R^{A} is H or alkyl (e.g. C₁₋₆ alkyl)); and [symbol]
represents an optional bond between two adjacent carbon atoms in the ring;
with the proviso that when Y and Z are both C═O, and R¹ and R⁸ together form a group NR¹³, at least one of R² and R³ is other than H or —CH₃.

2. A compound for use as claimed in claim 1, wherein said compound is of formula (Ia): 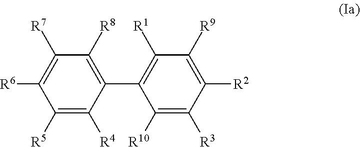
wherein R¹ to R¹⁰ are as defined in claim 1.

3. A compound for use as claimed in claim 1 or claim 2, wherein R⁹ is —O—C₁₋₆ alkyl, preferably —O—C₁₋₃ alkyl, e.g. —O—CH₃.

4. A compound for use as claimed in any one of claims 1 to 3, wherein R¹⁰ is selected from —OH and —O—C(O)R^{A} in which R^{A} is H or C₁₋₆ alkyl, preferably C₁₋₃ alkyl, e.g. methyl.

5. A compound for use as claimed in claim 1, wherein said compound is of formula (Ib): 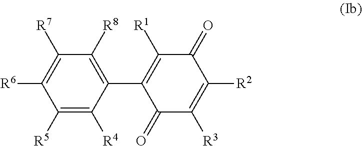
wherein R¹ to R⁸ are as defined in claim 1, subject to the proviso that when R¹ and R⁸ together form a group NR¹³, at least one of R² and R³ is other than H or —CH₃.

6. A compound for use as claimed in claim 1, wherein said compound is of formula (Ia′): 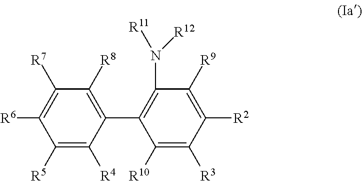
wherein each of R² to R¹² are as defined in any one of claims 1, 3 and 4.

7. A compound for use as claimed in claim 6, wherein R¹¹ and R¹² are both H.

8. A compound for use as claimed in claim 6, wherein R¹¹ is H and R¹² is a group of the formula —C(O)R^{A} (wherein R^{A} is H or alkyl, preferably C₁₋₆ alkyl, more preferably C₁₋₃ alkyl, e.g. methyl).

9. A compound for use as claimed in any one of the preceding claims, wherein R⁸ is H.

10. A compound for use as claimed in claim 1, wherein said compound is of formula (Ia″): 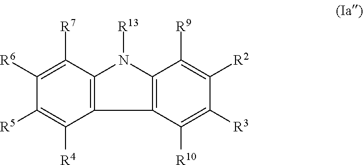
wherein each of R² to R⁷, R⁹, R¹⁰ and R¹³ are as defined in any one of claims 1, 3 and 4.

11. A compound for use as claimed in claim 10, wherein R¹³ is H, or a group of the formula —C(O)R^{A} in which R^{A} is H or alkyl, preferably C₁₋₆ alkyl, more preferably C₁₋₃ alkyl, e.g. methyl.

12. A compound for use as claimed in any one of the preceding claims, wherein R² is alkyl, preferably C₁₋₆ alkyl, more preferably C₁₋₃ alkyl, e.g. methyl.

13. A compound for use as claimed in any one of the preceding claims, wherein R³ is —O-alkyl, preferably —O—C₁₋₆ alkyl, more preferably —O—C₁₋₃ alkyl, e.g. —OCH₃.

14. A compound for use as claimed in any one of the preceding claims, wherein R⁴, R⁶ and R⁷ are each H.

15. A compound for use as claimed in any one of the preceding claims, wherein R⁵ is H or —O-alkyl, preferably —O—C₁₋₆ alkyl, more preferably —O—C₁₋₃ alkyl, e.g. —O—CH₃.

16. A compound for use as claimed in claim 1 which is selected from the following compounds and their pharmaceutically acceptable salts:

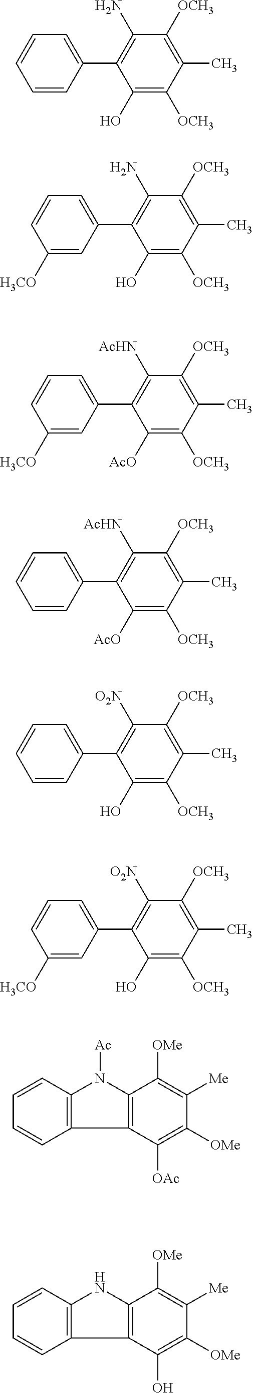

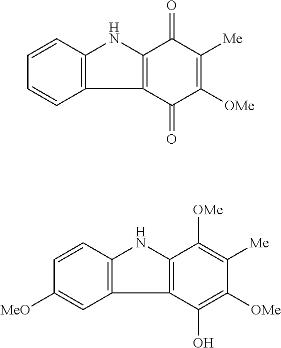 [SMILES: COC1=C(C)C(=O)c2[nH]c3ccccc3c2C1=O ; COc1ccc2[nH]c3c(OC)c(C)c(OC)c(O)c3c2c1]

17. A compound as claimed in any one of claims 1 to 16 for use in the treatment or prevention of leukaemia, for example chronic myeloid leukaemia (CML), acute myeloid leukaemia (AML), acute lymphocytic leukaemia (ALL) or t-ALL (T-cell acute lymphoblastic leukaemia).

18. A compound as defined in any one of claims 1 to 16, a stereoisomer, or a pharmaceutically acceptable salt thereof, for use in therapy or for use as a medicament.

19. A pharmaceutical composition comprising a compound as defined in any one of claims 1 to 16, a stereoisomer, or a pharmaceutically acceptable salt thereof, together with one or more pharmaceutically acceptable carriers, excipients or diluents.

20. Use of a compound as defined in any one of claims 1 to 16, a stereoisomer, or a pharmaceutically acceptable salt thereof, in the manufacture of a medicament for use in the treatment or prevention of leukaemia.

21. A method of treatment or prevention of leukaemia, said method comprising the step of administering to a subject in need thereof (e.g. a human patient) a pharmaceutically effective amount of a compound as defined in any one of claims 1 to 16, a stereoisomer, or a pharmaceutically acceptable salt thereof.
